(19) United States
(12) Patent Application Publication
(10) Pub. No.: US 2021/0145903 A1
(43) Pub. Date: May 20, 2021

(54) Lactobacillus Plantarum LP33 and Use Thereof in Preparation of Product for Promoting Lead Excretion

(71) Applicant: Southwest University, Chongqing (CN)

(72) Inventor: Huayi Suo, Chongqing City (CN)

(73) Assignee: Southwest University, Chongqing (CN)

(21) Appl. No.: 16/950,130

(22) Filed: Nov. 17, 2020

(30) Foreign Application Priority Data
Nov. 19, 2019 (CN) 201911131498.4

(51) Int. Cl. A61K 35/747; C12R 1/25; A61P 39/02; A23L 33/135; A23L 33/00

(52) U.S. Cl. CPC A61K 35/747; C12R 1/25; A61P 39/02; A23Y 2220/67; A23L 33/40; A23V 2002/00; A23L 33/135

(57) ABSTRACT

The disclosure provides Lactobacillus plantarum LP33 with an accession number of CCTCC NO: M 2019594, and use thereof in the preparation of a health product, a food and a medicament for promoting lead excretion. The disclosure is of important practical significance for developing functional health products, enriching types of fermentation products and establishing a Lactobacillus strain resource library, and brings new hope for preventing lead poisoning.

### TECHNICAL FIELD

The disclosure relates to a Lactobacillus plantarum strain for promoting lead excretion and use thereof in a food, a health product, and a medicament.

### BACKGROUND

Lead (Pb) is a non-essential toxic heavy metal element present in nature and can cause a number of acute and chronic diseases. Lead is widely used in the manufacture of lead-acid batteries, wire sheaths, lead pipes, chemical reaction vessels, pigments, and pesticides. Approximately millions of tons of lead are consumed worldwide every year, less than 25% of which will be recycled, and the remaining 75% of which can enter the environment through waste water, waste gas, waste residue, etc., causing pollution, harming animals, plants and human health. Most of the lead and compounds thereof are infected through the respiratory and digestive tracts, and a small part are infected through the skin. Approximately 40% of the lead is absorbed through the respiratory tract. After being absorbed, transported and redistributed into the human body, lead accumulates in different organs, resulting in an increase in lead load. Eventually 90%-95% of lead is mainly deposited in bones, and the half-life in bones may reach 25 years. Toxicity affects a plurality of systems, involving nervous, hematopoietic, vascular, immune, hepatic, renal, and skeletal systems. Children in growth and development are particularly sensitive to lead exposure, and injuries caused by lead exposure cannot be completely repaired.

Lead is excreted from the human body mainly from the following three ways: Firstly, the lead is excreted with the urine through kidneys, accounting for approximately 60% of the total excretion. Secondly, 30% of the lead is excreted into the intestinal cavity through bile secretion, and then excreted with the stool. Thirdly, approximately 8% of the lead (present in hair, nails and teeth) is discharged from the body through the loss of hair, nails and teeth. In 1978, MARCEL E. et al. proved that lead was mainly excreted through feces and urine by the radioactive element tracking method. Notably, they have also found that bile is the main way of intestinal lead excretion. Enterohepatic circulation controls the storage and reabsorption of 331 endogenous substances (e.g., bile acid (BA) and steroids) and xenobiotics (e.g., heavy metals and drugs) in the body. The Pb accumulated in the liver is released into the bile and re-secreted into the intestine, where most of the toxic metals are reabsorbed and re-engaged in the intestinal circulation, which will further aggravate the body damage caused by lead exposure. In recent years, a number of interesting studies and reviews have discussed the potential role of probiotics in BA metabolism.

Lactobacilli are considered to be non-pathogenic, safe-grade microorganisms, which participate in numerous metabolic activities in vivo, and have important probiotic properties. The cell wall components of lactobacilli as typical Gram-positive bacteria are principally composed of peptidoglycan, teichoic acid, polysaccharides and proteins. These cell wall components play a key role in the process of binding to heavy metals. A dense network structure composed of peptidoglycan and phosphoric acid is conducive to the adsorption of heavy metal ions. The proteins and polysaccharides on the surface of Lactobacillus cells contain functional groups such as —COOH, —NH₂, —SH, and —OH, which can effectively complex with heavy metal cations by surface complexation. As food grade probiotics, lactobacilli have attracted more and more attention to heavy metal adsorption characteristics thereof. If lactobacilli are added to heavy metal-containing foods as heavy metal adsorbents to remove heavy metals, or taken into the body as a dietary supplement, lactobacilli should be combined with heavy metals before the intestine absorbs them, and the heavy metals can be eliminated from the body through feces. Thus, toxic damage of heavy metals to the body can be inhibited. In addition, if lactobacilli are able to block the enterohepatic circulation of heavy metals, reduce the accumulation of heavy metals in tissues and promote the excretion of lead in the intestine, this will further reduce the toxic damage of heavy metals in the body. Therefore, lactobacilli have great potential as heavy metal adsorbents.

### SUMMARY

The objective of the disclosure is to provide a L. plantarum strain which can effectively promote lead excretion and use thereof in the preparation of a medicament, a food, and a health product.

Another objective of the disclosure is to provide a lead ion adsorbent, a pharmaceutical composition, a food, a health product, and a food additive containing the above-mentioned L. plantarum.

To achieve the above objectives, the disclosure provides the following technical solutions:

The disclosure provides a L. plantarum LP33 with an accession number of CCTCC NO: M 2019594.

The disclosure further provides use of the L. plantarum LP33 in the preparation of a medicament for promoting lead excretion.

The disclosure further provides use of the L. plantarum LP33 in the preparation of a food for promoting lead excretion.

The disclosure further provides use of the L. plantarum LP33 in the preparation of a health product for promoting lead excretion.

The disclosure further provides a pharmaceutical composition for promoting lead excretion, which contains a pharmaceutically effective dose of the L. plantarum LP33 with an accession number of CCTCC NO: M 2019594.

The disclosure further provides a food for promoting lead excretion, which contains the L. plantarum LP33 with an accession number of CCTCC NO: M 2019594.

The disclosure further provides a food additive for promoting lead excretion, which contains L. plantarum LP33 with an accession number of CCTCC NO: M 2019594; The disclosure further provides a health product for promoting lead excretion, which contains the L. plantarum LP33 with the accession number of CCTCC NO: M 2019594.

The disclosure further provides a lead ion adsorbent, which contains the L. plantarum LP33 with the accession number of CCTCC NO: M 2019594.

The disclosure selects 37 strains from a laboratory strain library for in vitro screening in accordance with the List of Strains Available for Food issued by the Ministry of Health in 2010. After a lead adsorption experiment, a simulated artificial gastric juice and bile salt tolerance test, and a strain antioxidant experiment, and by a comprehensive comparison, L. plantarum LP33 is the most targeted strain that has the potential to alleviate the toxic damage of lead. The lead ion clearance rate is 55.63% in a 50 mg/L lead ion solution, the survival rate in pH 3.0 artificial gastric juice is 104.08%, and the growth rate in 0.30% bile salt is 20.86%, and the strain has an excellent antioxidant effect. Scanning electron microscopy (SEM) finds that the surface of a bacterial cell can adsorb lead ions; transmission electron microscopy (TEM) reveals that in addition to the adsorption of lead ions on the surface of the bacterial cell, some lead ions enter the bacterial cell.

The disclosure further investigates the effect of L. plantarum LP33 on lead excretion in chronically lead-exposed rats. It is found that: treatment with L. plantarum LP33 significantly reduces blood lead in rats exposed to lead, and significantly increases lead content in feces; the total bile acid content in liver tissue and feces is significantly higher than that in the model group, which upregulates the mRNA expression of Fxr, Fgf15, Asbt, and Ostα in ileum tissue and of Cyp7a1, Cyp8b1, Mrp2, and Bsep in liver tissue, and down-regulates the mRNA expression of Shp, Ntcp, Fxr, and Fgfr4 in liver tissue. These results indicate that oral L. plantarum LP33 can induce liver BA synthesis and increase fecal BA excretion by down-regulating the FXR-FGF15 axis. This regulation can in turn cut off the enterohepatic circulation of the lead and enhance the excretion of lead in the feces. Therefore, L. plantarum LP33 can be used to prepare health foods and medicaments for promoting lead excretion.

The disclosure has the following beneficial effect: the disclosure provides use of L. plantarum LP33 in health products, foods and medicaments for promoting lead excretion, which not only expands the application range of L. plantarum LP33 and improves utilization value thereof, but also brings new hope for preventing lead poisoning.

### DEPOSIT OF BIOLOGICAL MATERIAL

Depository: China Center for Type Culture Collection (CCTCC); Address: Wuhan University, Wuhan, China; Deposit Date: Aug. 1, 2019; Deposit Number: CCTCC NO: M 2019594; Taxonomy and Name: L. plantarum LP33.

### BRIEF DESCRIPTION OF THE DRAWINGS

FIG. 1 illustrates the colony morphology and Gram's staining results of isolated strains.

FIG. 2 illustrates the testing the adsorption capacity of the strain for lead ions at lead ion conditions of 50 mg/L (A) and 500 mg/L (B).

FIG. 3 illustrates API 50CH reaction results of L. plantarum LP33.

FIG. 4 illustrates results of scanning electron microscopy with energy dispersive X-ray spectrometry (SEM/EDX) of lead adsorption of L. plantarum LP33.

FIG. 5 illustrates transmission electron microscopy (TEM) results of lead adsorption of L. plantarum LP33.

FIG. 6 illustrates the effect of L. plantarum LP33 on blood lead levels in chronically lead-exposed rats.

FIG. 7 illustrates the effect of L. plantarum LP33 on fecal lead levels in chronically lead-exposed rats.

FIG. 8 illustrates the effect of L. plantarum LP33 on the content of TBA in the liver tissues and feces of chronically lead-exposed mice.

FIG. 9 illustrates effects of L. plantarum LP33 on the mRNA expression of Fxr, Fgf15, Asbt, and Ostα in ileum tissue (A) and Cyp7a1, Cyp8b1, Mrp2, Bsep, Shp, Ntcp, Fxr, and Fgfr4 in liver tissue (B).

FIG. 10 illustrates the mechanism of L. plantarum LP33 promoting lead excretion.

### DETAILED DESCRIPTION

In order to make the objectives, technical solutions and advantages of the disclosure clearer, the preferred examples of the disclosure will be described in detail below with reference to the accompanying drawings.

I. Screening of Lactobacilli Adsorbing Lead Ions and Investigation of Adsorption Mechanism Thereof

1 Experimental Materials

In accordance with the List of Strains Available for Food issued by the Ministry of Health, this study selected 37 strains from the strain bank of the Suohuayi Laboratory, College of Food Science, Southwest University for subsequent experiments. The strains of this strain bank were isolated from traditional fermented dairy products in plateau regions such as Hongyuan, Qinghai, and Xinjiang, and traditional fermented vegetables in Sichuan and Chongqing region.

2 Experimental Methods

2.1 Activation and Culture of Lactobacilli

A strain deposited in a laboratory ampule tube was seeded in MRS Broth, and incubated in a 37° C. incubator for 18 h, and then seeded in MRS Broth in a ratio of 2% (v/v) for activation. Gram's staining was used for morphological observation. The strain was passaged twice before being used in subsequent experiments.

2.2 PCR Amplification of 16S rDNA Sequence

Bacterial Genomic DNA Extraction Kit was used to extract the DNA of the strain. PCR amplification was performed using a 25 μL reaction system, and detection was performed by agarose gel electrophoresis after the reaction was completed. Qualified samples were submitted to BGI Tech Solutions Co., Ltd. for sequencing, and sequencing results were analyzed by homology comparison using the BLAST program in NCBI.

2.3 Determination of Lead Adsorption Capacity of Strains

After all the activated strains were expanded and cultured at 37° C. for 18 h, and centrifuged for 10 min at 8,000×g to obtain bacterial cells. The bacterial cells were washed with sterilized ultrapure water twice, and the centrifugation process was repeated to further obtain Lactobacillus cells. A given amount of lead acetate was weighed and dissolved in ultrapure water to make a final lead ion concentration of 50 mg/L, and cells were filtered and removed with a 0.22 μm filter head. The cells obtained by centrifugation were resuspended in the above-mentioned lead solution so that the concentration of lactobacilli therein reached 1 g/L of wet cells. A bacterial suspension obtained above was adjusted to pH 6.0, incubated at 37° C. under shaking for 1 h, and then centrifuged for 10 min at 8,000×g. Supernatant was obtained and lead concentration thereof was measured using an atomic absorption spectrophotometer. The adsorption capacity of lactobacilli for lead ions can be calculated by the following formula:

$$Adsorption of lead ions ( \% ) = \frac{{C}_{0} - {C}_{1}}{{C}_{0}} \times 1 \; 00 \% , \qquad ( 1 )$$

where:

C₀—concentration of lead ion in initial solution

C₁—concentration of lead ion in the solution after strain adsorption

According to the result of the adsorption capacity of the strains under the condition of 50 mg/L lead ion, lactobacilli with a lead ion adsorption capacity of >50% were selected from the 37 strains of lactobacilli; eight primarily screened strains were resuspended at a lead ion concentration of 500 mg/L for secondary screening to further evaluate the adsorption capacity thereof.

2.4 Determination of Tolerance of Strains to Simulated Gastrointestinal Fluid

2.4.1 Determination of the Survival Rate of Strains in pH 3.0 Artificial Gastric Juice

Isolated strains were cultured for 18 h at 37° C., and centrifuged for 15 min at 3,000 r/min to collect bacterial cells; the bacterial cells were washed with sterile normal saline and resuspended as a bacterial suspension. The resulting bacterial suspension was mixed with artificial gastric juice (0.2% NaCl and 0.35% pepsin in a 1:10,000 ratio, adjusted to pH 3.00 with 1 mol/L HCl) at a volume ratio of 1:9; after incubation for 3 h at 37° C., viable cell counts at 0 and 3 h were determined by the spread plate method, and the survival rate of the strain in pH 3.00 artificial gastric juice was calculated according to formula (1).

$$c = \frac{{m}_{1}}{{m}_{2}} \times 100 \% , \qquad ( 2 )$$

where:

c—survival rate, in %;

m₁—viable cell count at 3 h, in CFU/mL;

m₂—viable cell count at 0 h, in CFU/mL.

2.4.2 Determination of the Growth Efficiency of the Stain in 0.3% Bile Salt.

The isolated strains were cultured for 18 h at 37° C., and inoculated with 2% inoculum size in MRS-THIO medium supplemented with 0.00% and 0.30% bovine bile salt; the growth rate of the isolated strains was measured after incubation for 24 h at 37° C. Using uninoculated liquid medium as a blank control, the growth rate of the strain in bile salt was calculated according to formula (2).

$$c = \frac{{A}_{2} - {A}_{0}}{{A}_{1} - {A}_{0}} \times 100 \% , \qquad ( 3 )$$

where:

c—growth rate, in %;

A₀—blank control OD600 nm value;

A₁—OD_{600 nm} value of the medium supplemented with 0.00% bile salt;

A₂—OD_{600 nm} value of the medium supplemented with 0.30% bile salt.

2.5 DETERMINATION OF ANTIOXIDANT CAPACITY OF LACTOBACILLI

Strains with excellent performance with a lead ion rate of 45% and a growth rate of >10% in 0.30% bile salt were selected for the next step of determining antioxidant capacity.

2.5.1 Preparation of Lactobacillus Samples

A cultured Lactobacillus solution was centrifuged for 20 min at 8,000×g to obtain bacterial cells, washed twice with PBS (pH=7.2), and then resuspended in PBS; the concentration of the bacterial suspension was adjusted to an OD₆₀₀ value of 1.0, and two 5 mL aliquots of the bacterial suspension were taken for future use. One aliquot was used as intact cells; the other was placed in an ice bath to sonicate the cells (ultrasonic horn ϕ6; ultrasound on for 4 s; ultrasound off for 4 s; power 46%) for 10 min, and centrifuged for 10 min at 8,000 r/min and 4° C. Supernatant was transferred to a sterile centrifuge tube to obtain a cell-free extract.

2.5.2 Determination of the 1,1-Diphenyl-2-Picrylhydrazyl (DPPH) Free Radical Scavenging Activity of Lactobacilli

According to the method described in references, 1 mL of 0.2 mmol/L DPPH-absolute alcohol solution was added to 1 mL each of a supernatant of a bacterial suspension and a cell-free extract and mixed well, and the reaction was performed in the dark for 30 min; the mixture was centrifuged for 10 min at 6,000 r/min and 4° C., and supernatant was pipetted and placed at a wavelength of 517 nm to measure the optical density (OD) value. PBS was used instead of a test sample solution as a control group.

$$DPPH free radical scavenging activity ( \% ) = \frac{D - {D}_{0}}{{D}_{0}} \times 100 \% , \qquad ( 4 )$$

where:

D—the OD value of the test sample;

D₀—the OD value of the blank control.

2.5.3 Determination of the Hydroxyl Radical Scavenging Activity of Lactobacilli

According to the literature, a supernatant of the bacterial suspension and a cell-free culture were uniformly mixed with 1 mL each of 2.5 mmol/L 1,10-phenanthroline monohydrate solution and sterile PBS (pH=7.4) 1 mL each, and then well mixed with 1 mL of 2.5 mmol/L Fe2SO4 solution and 1 mL of 20 mmol/L H₂O₂ solution, and the mixture was placed in a 37° C. water bath for 1 h. After completion of the water bath, the mixture was centrifuged for 10 min at 8,000 r/min, and supernatant was pipetted and placed at a wavelength of 536 nm to measure the OD value. PBS was used instead of a test sample solution as a blank control, and instead of a H₂O₂ solution as a negative control. The calculation formula is as follows:

$$Hydroxyl radical scavenging activity ( \% ) = \frac{H - {H}_{0}}{{H}_{1 - {H}_{0}}} \times 100 \% , \qquad ( 5 )$$

where:

H—the OD value of the test sample;

H₀—represents the OD value of the blank control;

H₁—represents the OD value of the negative control.

2.5.4 Determination of the Reducing Ability of Lactobacilli

Naught point five milliliter (0.5 mL) of test sample was mixed well with 0.5 mL of PBS (0.2 mol/L, pH=6.6) and 0.5 mL of potassium ferricyanide (mass fraction 1%), placed in a 50° C. constant temperature water bath for 20 min, and cooled in ice water. Then 0.5 mL of trichloroacetic acid (mass fraction 10%) was added; the mixture was centrifuged for 10 min at 4,000 rpm; 1 mL of supernatant was pipetted and mixed well with 1 mL of distilled water and 1 mL of ferric chloride (mass fraction 0.1%); after reaction for 10 min, the OD value thereof was measured at a wavelength of 700 nm; PBS was used instead of the test sample as a blank control.

$$Reducing ability ( \% ) = \frac{{A}_{S} - {A}_{0}}{{A}_{0}} \times 100 \% \qquad ( 6 )$$

where:

A_{S}—the OD value of the test sample;

A₀—the OD value of the blank control.

Based on the above results, lactobacilli with excellent lead adsorption capacity, simulated gastrointestinal fluid tolerance capacity, and in vitro antioxidant capacity were selected for subsequent tests.

2.6 Identification by API Kit

Isolated strains were cultured for 18 h at 37° C., and centrifuged for 15 min at 3,000 r/min to collect bacterial cells; the bacterial cells were washed with sterile normal saline and resuspended as a bacterial suspension. Refer to the instructions of the API kit for operation.

2.7 SEM/EDX of L. plantarum LP33 Before and after Adsorption of Lead Ions

The experiment of LP33 adsorption of lead ions was carried out according to the method in 2.3. Centrifuged bacterial cells were fixed in 2.5% glutaraldehyde solution at 4° C. overnight. After fixation, the sample was rinsed thrice with 0.1 M PBS (pH 7.0) for 15 min each time; the sample was fixed with 1% osmic acid solution for 1-2 h; the osmic acid waste solution was removed carefully, and the sample was rinsed thrice with 0.1 M PBS (pH 7.0) for 15 min each time; the sample was dehydrated with different gradient concentrations (including 30%, 50%, 70%, 80%, 90%, and 95%) of ethanol solution, and each concentration was treated for 15 min; then the sample was treated with 100% ethanol twice for 20 min each time. The sample was treated with a mixture of ethanol and isoamyl acetate (VN=1/1) for 30 min, followed by treating with pure isoamyl acetate for 1 h or standing overnight. After critical point drying, the sample was coated before observation. A well-treated sample was observed under a scanning electron microscope.

Except that no lead ion adsorption experiment was required, the sample preparation of the blank control was also carried out according to the above method. A scanning electron microscope (SEM) was used to observe changes in cell morphology in the sample, and elemental composition was analyzed with an energy dispersive spectrometer (EDX) connected thereto.

2.8 TEM Observation of L. plantarum LP33 Before and after Adsorption of Lead Ions

The experiment of LP33 adsorption of lead ions was carried out according to the method in 2.3. Centrifuged bacterial cells were fixed in 2.5% glutaraldehyde solution at 4° C. overnight. After fixation, the fixing solution was decanted and the sample was rinsed thrice with 0.1 M PBS (pH 7.0) for 15 min each time; the sample was fixed with 1% osmic acid solution for 1-2 h; the osmic acid waste solution was removed carefully, and the sample was rinsed thrice with 0.1 M PBS (pH 7.0) for 15 min each time; the sample was dehydrated with different gradient concentrations (including 30%, 50%, 70%, 80%, 90%, and 95%) of ethanol solution, and each concentration was treated for 15 min; then the sample was treated with 100% ethanol twice for 20 min each time; finally, the sample was treated with acetone for 20 min. The sample was successively treated with a mixture of embedding agent and acetone (VN=1/1) for 1 h, a mixture of embedding agent and acetone (VN=3/1) for 3 h, and pure embedding agent overnight; an osmosis-treated sample was embedded and heated at 70° C. overnight to obtain an embedded sample. The sample was sectioned in an ultramicrotome to obtain 70-90 nm sections. The sections were stained with lead citrate solution and saturated uranyl acetate in 50% ethanol for 5-10 min, respectively, and then observed under a transmission electron microscope.

Except that no lead ion adsorption experiment was required, the sample preparation of the blank control was also carried out according to the above method. An SEM was used to observe changes in cell morphology in the sample.

2.9 Statistical Analysis

Three parallel tests were performed for each test. The test data were expressed as “mean±standard deviation (SD)”. SPSS20 was used for analysis of variance (ANOVA), and P<0.05 indicated statistical significance.

3 Results and Analysis

3.1 Colony Morphology and Cell Morphology of Strains

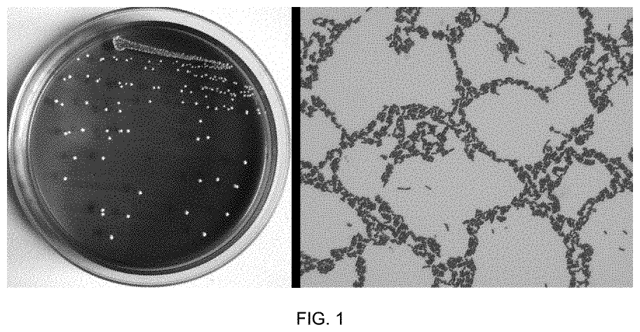

Thirty-seven activated strains formed single colonies in an MRS medium. The colonies had almost the same morphology, most of which appeared round, white, and smooth and moist on the surface. After Gram's staining, purple cell morphology was observed microscopically and was determined to be Gram-positive (G+). Among them, the colony morphology and Gram's staining result of strain 33 are shown in FIG. 1.

3.2 PCR Amplification Results of Strain 16S rDNA Sequence

A PCR amplified sequence of strain 33 was identified as L. plantarum by Blast alignment in NCBI.

3.3 Adsorption Capacity of Lactobacilli for Lead Ions

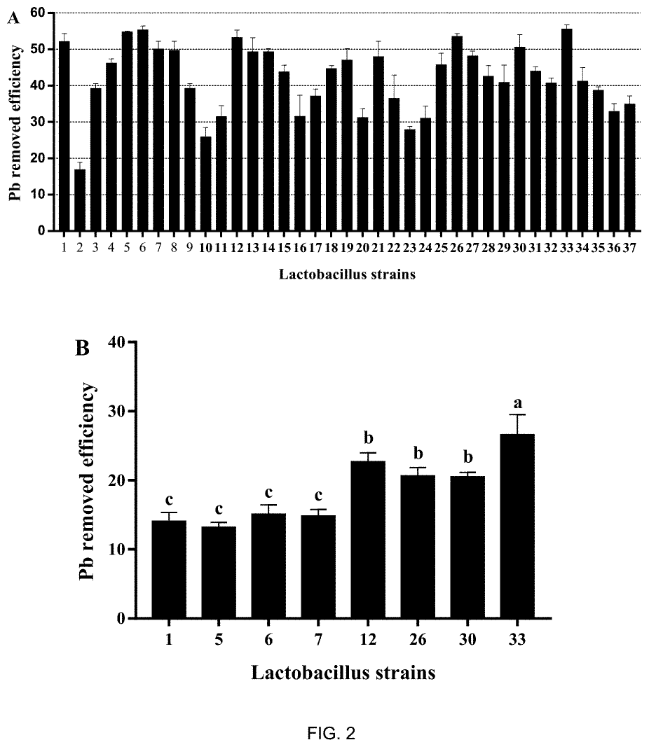

When screening lactobacilli that can alleviate lead toxicity, it should first be considered that the lactobacilli should have a strong lead ion adsorption capacity, so that the lactobacilli can allow lead ions to be adsorbed first and then excreted from the body with feces before the lead ions enter the host intestine to be absorbed. Thus, this lowers the absorption of lead ions in the intestine, and thereby reduces the accumulation of lead ions in various organs of the host. The adsorption capacity of 37 experimental Lactobacillus strains for lead ions was expressed in terms of lead ion adsorption rate (%). As shown in FIG. 2A, when the concentration of lead ions is 50 mg/L, there is a large difference in the ability of different strains to adsorb lead ions (from 16.93% to 55.63%). Under this condition, strains with an adsorption rate greater than 50% were selected for secondary screening, which were: Lactobacilus fermentum strains 1, 5, 6, and 12 and L. plantarum strains 7, 26, 30, and 33. The results show that when the initial concentration of lead ions increases to 500 mg/L, the aluminum ion adsorption capacity of these eight strains of lactobacilli generally decreases (FIG. 2B), but L. plantarum LP33 still shows the strongest lead ion adsorption capacity (26.83%).

3.4 Tolerance of Lactobacilli to Simulated Gastrointestinal Fluid

One of the main functions of the human gastrointestinal fluid is to bear the digestion of food and the absorption of nutrients in the human body. Secondly, a large amount of enzymes and bile salts contained therein can destroy the cell membrane structure and is the first barrier to prevent microorganisms from entering the gastrointestinal tract. Therefore, an ideal functional strain should have good acid and bile salt resistance, which can ensure that the strain can smoothly pass through the acid environment of the stomach to reach the intestine and continue to maintain the activity in the intestine. In this study, according to the pH values and bile salt concentrations of gastric juice and intestinal juice in normal human physiological environment, as well as the residence time of food in the stomach and intestine, pH 3.0 artificial gastric juice and 0.3% bile salt concentration were selected to test the tolerance of lactobacilli to screen out strains with better tolerance. The results showed that: strain 33 showed good bile salt/gastric acid resistance, and the survival rate thereof in artificial gastric juice reached 104.08%, the growth rate thereof in 0.30% bile salt reached 20.86%; the survival rate of strain 2 in artificial gastric juice was 78.69%, the growth rate thereof in 0.30% bile salt was only 5.56%.

3.5 Antioxidant Ability of Lactobacilli

3.5.1 DPPH Radical Scavenging Activity of Lactobacilli

DPPH is a stable nitrogen-centered synthetic free radical. When a free radical quencher or an antioxidant reacts with DPPH, a solution will change from deep violet to light yellow or colorless. Therefore, the change in OD value at 517 nm can be measured to quantitatively detect the free radical scavenging in the sample, and thus evaluate the free radical scavenging ability of the test sample. The DPPH radical scavenging activity of the complete cell suspension of strain 33 was 23.20%, which was higher than that of the cell-free extract. For the cell-free extract, strain 33 showed the highest DPPH radical scavenging activity (13.63%).

3.5.2 Hydroxyl Radical Scavenging Activity of Lactobacilli

Hydroxyl radical (.OH) is a kind of strong oxidizing reactive oxygen species (ROS), which can destroy the permeability of biological cell membrane, and lead to oxidative damage of DNA, destroying normal cell functions. Therefore, the hydroxyl radical scavenging activity is an important indicator for evaluating the antioxidant activity of lactobacilli. The complete cell suspension of strain 33 showed the strongest hydroxyl radical scavenging activity (30.81%). For the cell-free extract, the hydroxyl radical scavenging activity of strain 33 was higher than 25%, significantly higher than that of other strains (P<0.05).

3.5.3 Reducing Ability of Lactobacilli

Reducing ability mainly refers to some redox reaction enzymes and some non-enzymatic compounds with antioxidant ability not only to inhibit the generation of ROS, but also to control the reaction of Fe²⁺ and other transition metal ions, thereby effectively preventing the generation of oxidation reactions, which is called reducting ability. Therefore, reducing ability is often selected as an index to evaluate the antioxidant capacity of a strain. Strain 33 has the strongest reducing ability; the reducing ability of the complete cell suspension or a cell-free extract is above 90%, which is significantly better than that of other strains (P<0.05).

3.6 Identification Results of Biochemical Characteristics of the Optimal Resistant Strain

By comprehensive comparison of the evaluation results of lead adsorption capacity, tolerance of artificial gastric juice and bile salts, and anti-oxidation of the strains, strain 33 is the optimal resistant strain. The phenotypic identification at the Lactobacillus species level is mainly based on carbohydrate fermentation tests. API 50 CH Kit is to identify the strain's utilization of 49 different carbohydrates.

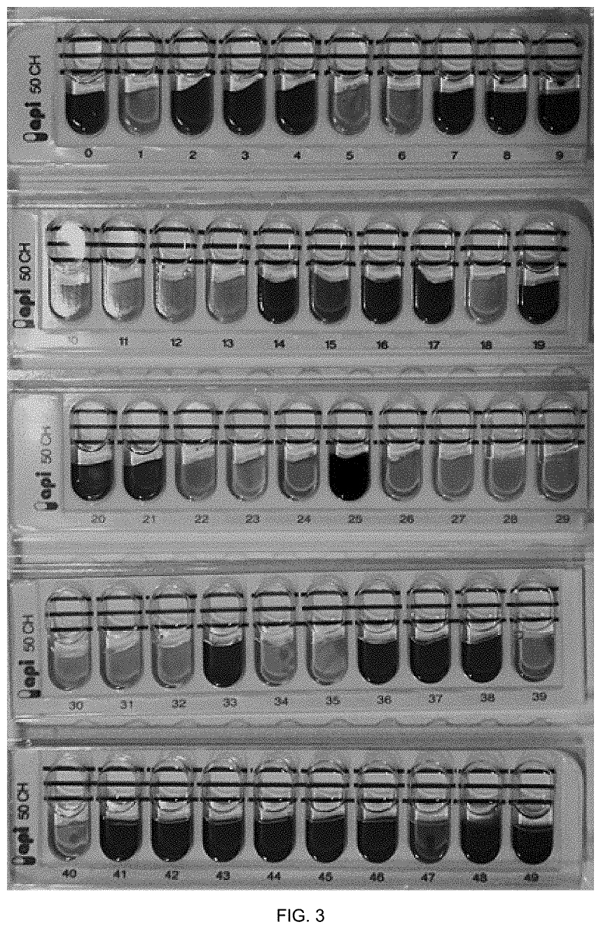

FIG. 3 shows the results of the API 50 CH reaction for strain 33. Table 1 shows the results of the fermentation tests of 49 carbohydrates of strain 33. As can be seen from FIG. 3 and Table 1, of the 49 carbon sources tested, strain 33 can utilize 25 of these carbohydrates. According to the final identification by the API lab plus system, strain 33 is L. plantarum, with an ID value of 95.00% and a T value of 0.36. The ID value thereof has not reached above 99.0%, suggesting that this strain is a new variant of L. plantarum.

TABLE 1
Results of the fermentation
tests of 49 carbohydrates of strain 33

| Tube | | Reaction |
|---|---|---|
| No. | Carbohydrate | result |
| 0 | Blank | − |
| 1 | Glycerol | + |
| 2 | Erythritol | − |
| 3 | D-arabinose | − |
| 4 | L-arabinose | − |
| 5 | D-ribose | + |
| 6 | D-xylose | + |
| 7 | L-xylose | − |
| 8 | D-adonitol 1 | − |
| 9 | Methyl β-D-xylopyranoside | − |
| 10 | D-galactose | + |
| 11 | D-glucose | + |
| 12 | D-fructose | + |
| 13 | D-mannose | + |
| 14 | L-sorbose | − |
| 15 | L-rhamnose | + |
| 16 | Dulcitol | − |
| 17 | Inositol | − |
| 18 | Mannitol | + |
| 19 | Sorbitol | − |
| 20 | Methyl-α-D-mannopyranoside | − |
| 21 | Methyl-α-D-glucopyranoside | − |
| 22 | N-acetyl-glucosamine | + |
| 23 | Amygdalin | + |
| 24 | Arbulin | + |
| 25 | Esculin and ferric citrate | + |
| 26 | Salicin | + |
| 27 | D-cellobiose | + |
| 28 | D-maltose | + |
| 29 | D-lactose | + |
| 30 | D-melibiose | + |
| 31 | D-sucrose | + |
| 32 | D-trehalose | + |
| 33 | Inulin | − |
| 34 | D-melezitose | + |
| 35 | D-raffinose | + |
| 36 | Starch | + |
| 37 | Glycogen | − |
| 38 | Xylitol | − |
| 39 | D-gentiobiose | + |
| 40 | D-Toulon sugar | + |
| 41 | D-Iyxose | − |
| 42 | D-tagatose | − |
| 43 | D-fucose | − |
| 44 | L-fucose | − |
| 45 | D-arbaitol | − |
| 46 | L-arbaitol | − |
| 47 | Potassium gluconate | + |
| 48 | 2-Keto-potassium gluconate | − |
| 49 | 5-Keto-potassium gluconate | − |

3.7 SEM/EDX of Lead Adsorption by LP33

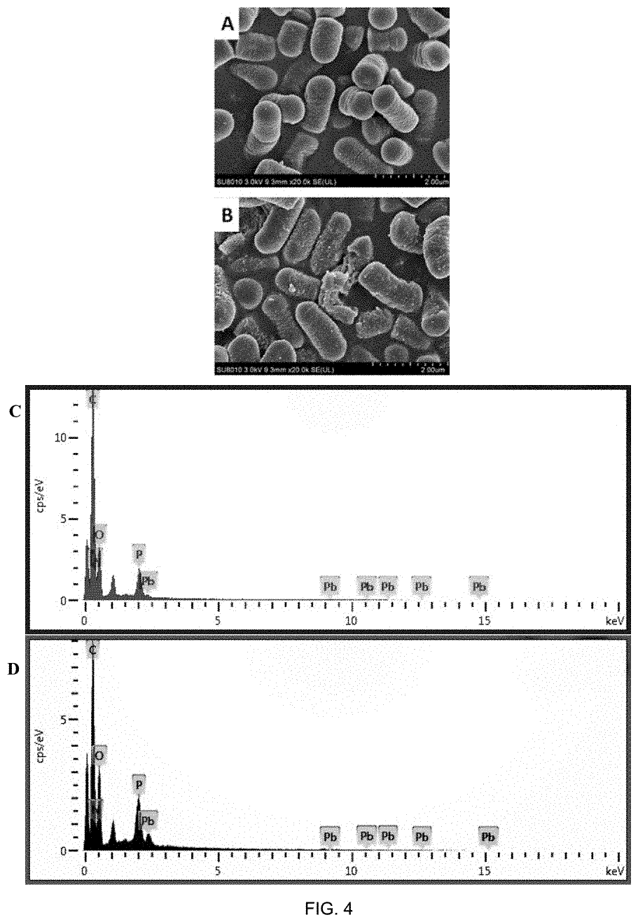

The SEM results are shown in FIG. 2. Compared with the normal blank control cells (FIG. 4A), some new irregular particles appeared on the surface of the cells after the lead ion adsorption test, and some particles showed aggregation. At the same time, it was also found that the rupture of bacterial cells occurred. EDX was used to scan some areas before and after the lead ion adsorption. The results showed that the scan spectrum of the bacterial cells in the lead ion exposure group was significantly higher than the lead element peaks in the blank control group (FIGS. 4C and D). As shown in Table 2, in the percentage of element atoms, the lead element in the lead ion exposure group accounted for 0.18%, while that in the blank control group accounted for only 0.03%. This result further verified the adsorption of lead ions by L. plantarum LP33 cells.

TABLE 2
Elemental atomic percentage of bacterial cells by
EDX before and after lead adsorption scanning

| | Atomic percentage (%) | |
|---|---|---|
| Element | Lead ion exposure group | Blank control group |
| C | 54.30 | 51.43 |
| N | 24.29 | 21.71 |
| O | 20.46 | 25.65 |
| P | 0.92 | 1.03 |
| Pb | 0.03 | 0.18 |
| Total | 100.00 | 100.00 |

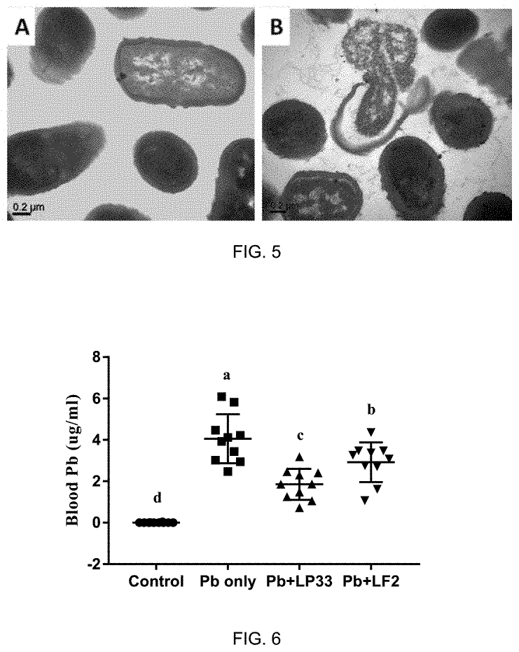

The TEM results are shown in FIG. 3. After the adsorption was completed, there were obvious deposits around the bacterial cells, and some deposits were found inside the protoplasts (FIG. 5B); but there was no similar deposits around the bacterial cells in the blank control group (FIG. 5A). This result is consistent with the SEM result.

II. Promotion of Lead Excretion in Lead Poisoning Rats by L. plantarum

1 Experimental Materials

Strain: L. plantarum LP33 (LP33) was deposited at the China Center for Type Culture Collection (CCTCC) (Accession Number: M 2018592) and College of Food Science, Southwest University (Accession Number: 33). Lactobacillus fermentum 2 (LF2) is deposited at the China General Microbiological Culture Collection Center (CGMCC) (Accession Number: 16637), College of Food Science, Southwest University (Accession Number: 2).

Experimental animals: 40 male Sprague-Dawley (SD) rats aged 4-6 weeks, purchased from the Laboratory Animal Center of Chongqing Medical University, license number: SCXK (Chongqing) 2018-0003.

2 Experimental Methods

2.1 Grouping and Treatment of Experimental Animals

TABLE 3
Design scheme of animal experiment

| Group | Treatment |
|---|---|
| Control (n = 10) | Ordinary water + normal saline (ig) |
| Pb only (n = 10) | Lead water + normal saline (ig) |
| Pb + LP33 (n = 10) | Lead water + normal saline (ig) + LP33 (ig) |
| Pb + LF2 (n = 10) | Lead water + normal saline (ig) + LF2 (ig) |

NOTE: Ordinary water: lead-free ordinary drinking water for rats to drink ad libitum; lead water: lead acetate trihydrate dissolved in drinking water to a concentration of 500 mg/L for mice to drink ad libitum; LP33, LF2: bacterial suspension with a concentration of 1×10⁹ CFU/mL, the intragastric dosage of which is determined by 0.1 mL per 100 g body weight; normal saline: the intragastric dosage is determined by 1 mL per 100 g body weight. The experiment lasts for eight weeks.

As shown in Table 3, 40 SD rats were randomly divided into four groups: normal group (control), model group (Pb only), strain 33 group (Pb+LP33), and strain 2 group (Pb+LF2). Each group contains 10 rats. The rats were acclimated for one week before the experiment began, and the experiment period was eight weeks. The feces of each rat were collected separately at regular intervals every week.

2.2 Sample Collection and Storage

At the end of the 8-week experiment, the rats were weighed after 18 hours of fasting and deprivation of water, and blood was drawn from eyeballs after ether anesthesia. Specifically, 2 mL of whole blood was used for detecting blood lead, which was put into heparin sodium tubes and mixed upside down for subsequent test. The remaining blood was centrifuged for 10 min at 3,000 r/min and 4° C. to collect serum, and stored at −80° C. for future use. After the blood was drawn, the rats were sacrificed by cervical dislocation, and the liver and ileum tissues of the rats were quickly dissected out, marked and stored at −80° C. for future use.

2.3 Determination of Fecal and Blood Lead Levels

Fecal and blood samples were transferred into an Erlenmeyer flask soaked with 20% nitric acid overnight, and 10 mL of concentrated nitric acid was added thereto; the mixture was left overnight and digested on an adjustable electric hot plate, until the digestive juice appeared colorless and transparent or slightly yellowish. After cooling, the digestive juice was diluted to a constant volume and a flame atomic absorption spectrophotometer was used to determine the lead levels of the samples.

2.4 Determination of Total Bile Acid (TBA) in Feces and Liver Tissues

The TBA of feces and liver tissue was measured in accordance with the instructions of the TBA Assay Kit.

2.5 Determination of mRNA Expression of Related Genes in Tissues by qRT-PCR

Total RNA was extracted from the ileum and liver in accordance with the instructions of Trizol; then 1 μL of RNA sample was mixed with 1 μL (oligo) primer dT and 10 μL of sterile ultrapure water, and the mixture was reacted for 5 min at 65° C. After the reaction was completed, 1 μL of Ribolock RNase Inhibitor, 2 μL of 100 mM dNTP Mix, 4 μL of 5× Reaction Buffer, and 1 μL of Revert Aid M-mu/v RT was added to the reaction system and mixed well, and cDNA was synthesized at 42° C. for 60 min and at 70° C. for 5 min; the purity and concentration of cDNA was measured by using an ultramicrospectrophotometer, and the cDNA concentration of each sample was adjusted to the same level (1 μg/pL). Next, target genes were reverse transcribed and amplified using primer sequences described in Table 7. The reaction conditions were: initial denaturation at 95° C. for 10 min; 40 cycles of 95° C. for 15 s, 60° C. for 1 min, and 72° C. for 30 s. Finally, using GAPDH as a reference gene, the relative expression of the target genes was calculated by 2^{-66 ΔCT}.

TABLE 4
Primer sequences

| Primer | | |
|---|---|---|
| name | Forward Primer | Reverse Primer |
| GAPDH | AAGTTCAACGGCACAGTCA | ACGCCAGTAGACTCCACGACAT |
| | AGG | |
| Cyp7a1 | GCTGAGGGATTGAAGCACA | GATGCCCAGAGAATAGCGAGG |
| | AGA | |
| Cyp8b1 | CAGATTTGACCTACTTTTC | CCCAACCAGTTACTTATGCCGT |
| | CCCA | |
| Shp | TCTTCCTGCTTGGGTTGGC | GAGGGTTGTGGTGGGTCTGG |
| Bsep | CCCTGTGAAGGCATGGTGA | GGATGTTTTCTGCGATAGTGGTG |
| | C | |
| Ntcp | TTACGGCTACCTCCTCCCT | TCCATGCTGATGGTGCGTCT |
| | GAT | |
| Mrp2 | CAGATGAGGAGGTTTGGAG | CAGGACCAGGATTTTGGATTTTC |
| | GG | |
| Abst | AACTTCAATGCCATTCTCA | TGACAGAGGAAGCCCACGAA |
| | GCAC | |
| Ostα | CAGCCCTCCATTTTCTCCA | CCAACCTTGTTATCTTTCTTTCG |
| | TC | A |
| Fxr | ATTTACAAGCCACGGACGA | GTTGGAATAATAGGACGAGGAGG |
| | GTT | A |
| Fgf15 | CCAACTGCTTCCTGCGTAT | CGAGTAGCGAATCAGCCCGTA |
| | CC | |
| Fgf4 | AACGAGGACCCCAAGACCC | TGGACAGCGGAATTTGACAGT |

2.6 Data Analysis

One-way ANOVA in SPSS 17.0 was used to perform a significance analysis (P<0.05), and the final result was expressed as mean±standard deviation (SD) (x±s). The graphs used herein are plotted by Graphpad software.

3 Experimental Results and Analysis

3.1 Effect of L. plantarum LP33 on Lead Content in Blood and Feces of Lead-Exposed Mice

The blood lead content in rats is shown in FIG. 6. The lead content of the lead exposure group was much higher than that of the control group. The blood lead content of the Pb+LP33 and Pb+LF2 groups was significantly lower than that of the model group, and the lead content of the Pb+LP33 group was significantly lower than that of the Pb+LF2 group (P<0.05). The results showed that L. plantarum LP33 significantly reduced the lead content in the blood of chronically lead-exposed mice (P<0.05).

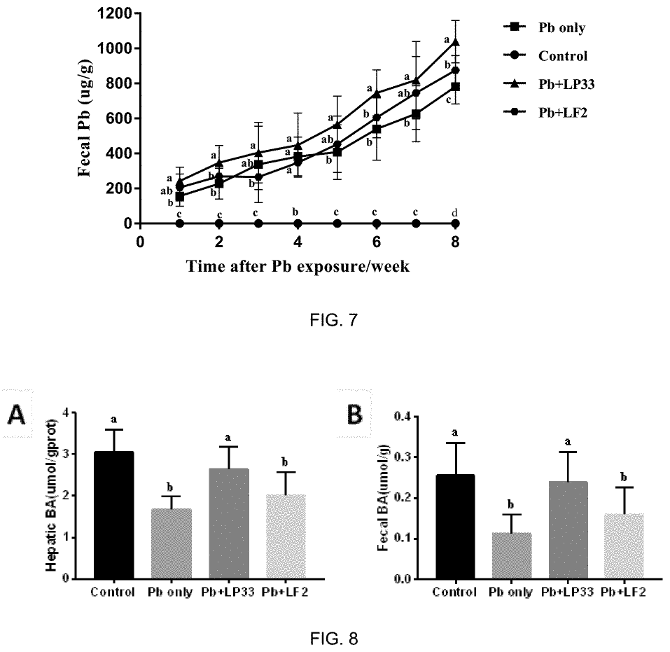

The change of lead content in rat feces throughout the experimental period is shown in FIG. 7. Feces in the normal group were almost lead-free throughout the period. In the other three groups of mice, when lead was exposed, the content of lead ions in feces increased significantly. Compared with the lead-exposed group, L. plantarum LP33 significantly increased the fecal lead content at each time point, and the lead excretion effect of L. fermentum 2 was significantly lower than that of L. plantarum LP33. It was found that L. plantarum significantly reduced the lead content in the weekly feces of chronically lead-exposed mice, and the lead excretion effect was significantly better than that of L. fermentum 2.

3.2 Effects of L. plantarum LP33 on the Content of TBA in Liver Tissues and Feces of Lead-Exposed Mice

Bile acid (BA) is the main component of bile. As a digestive fluid, BA can promote the digestion and absorption of lipids and can also serve as an excretory fluid to convert in vivo metabolites such as cholesterol into non-nutrients through the bioconversion of the liver, which are passed through the intestinal cavity and excreted from the body with feces. Some studies have demonstrated the potential mechanism of BA in stimulating lead excretion. Small aggregates of dihydroxy bile salts react with divalent heavy metal ions (such as Pb²⁺ and Cd²⁺) and form sparingly soluble complexes.

As shown in FIG. 8, after administration of L. plantarum LP33 by gavage, the content of BA in liver tissues and feces of chronically lead-exposed mice was significantly higher than that in the model group (P<0.05). However, the content of BA in liver tissues and feces of the L. fermentum group 2 increased slightly compared with the model group, but there was no significant difference. This showed that L. plantarum LP33 promoted the synthesis and excretion of bile acids, and the effect was better than that of strain 2.

3.3 Effect of L. plantarum LP33 on mRNA Expression in Liver Tissues of Lead-Exposed Mice

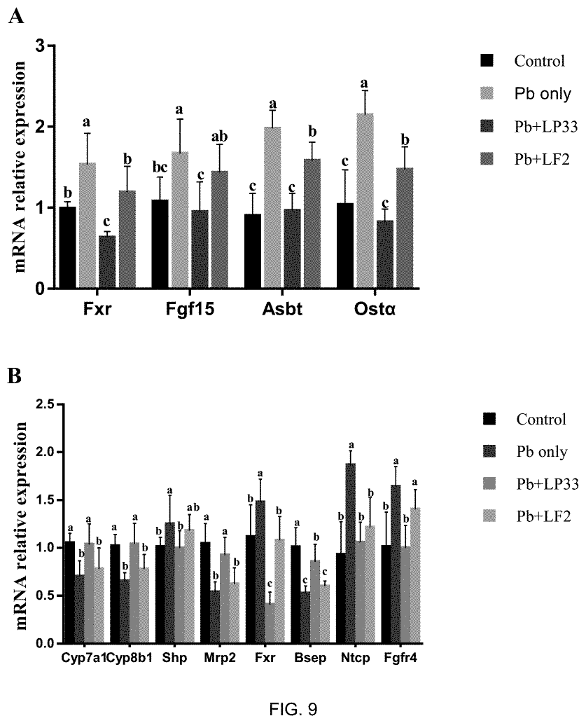

The enterohepatic circulation of BA plays important roles in host lipid metabolism, glucose homeostasis, liver bile formation and intestinal function. BA also regulates the storage and reuse of heavy metals (such as lead, cadmium, and mercury) because they are released from the liver through the bile into the intestinal lumen, where they are reabsorbed and transported back to the liver. Reportedly, FXR-FGF15 axis plays an important role in BA homeostasis. The BA can activate FXR and further the expression of organic solute transporter (Ostα), which is related to the basolateral secretion of the BA in the ileum. FXR activation also upregulates ileal Fgf15 expression, which in turn signals the liver to inhibit the expression of rate-limiting enzymes such as 7α-hydroxylase (Cyp7a1) and sterol-12α-hydroxylase (Cyp8b1), thereby inhibiting liver BA synthesis. The ileal mRNA expression level of Ostα and apical sodium-dependent bile acid transporter (Asbt) induced by oral L. plantarum LP33 was significantly lower than that of the model group (P<0.05) (FIG. 9A). In addition, L. plantarum LP33 treatment significantly increased the mRNA expression of Cyp7a1, Cyp8b1, bile salt export pump (Bsep) and multidrug resistance-associated protein 2 (Mrp2) in the liver, while significantly down-regulating the expression of liver small heterodimer partner (Shp) and sodium taurocholate cotransporting polypeptide (Ntcp) (P<0.05, FIG. 9B). The role of L. plantarum LP33 in inducing Pb excretion depends in part on the FXR-FGF15 gut-liver axis. Oral administration of L. plantarum LP33 significantly inhibited ileal mRNA expression of fibroblast growth factor 15 (Fgf15) and farnesoid X receptor (Fxr) (P<0.05, FIG. 9A), and also significantly inhibited the mRNA expression of Fxr and fibroblast growth factor receptor 4 (Fgfr4) in liver tissue (P<0.05, FIG. 9B).

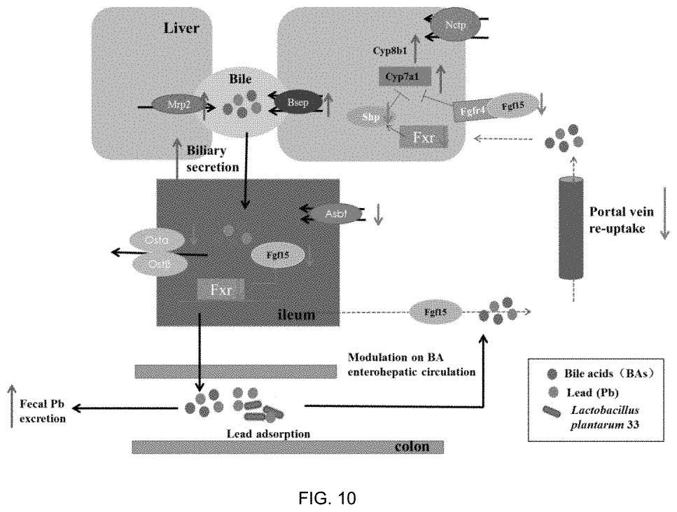

Taken together, these results indicate that oral L. plantarum LP33 can induce liver BA synthesis and increase fecal BA excretion by down-regulating the FXR-FGF15 axis. This regulation can in turn cut off the enterohepatic circulation of lead and enhance the fecal lead excretion. The specific mechanism is shown in FIG. 10.

Lastly, the above examples are only used to illustrate the technical solutions of the disclosure and not to limit them. Although the disclosure has been described by referring to the preferred examples of the disclosure, those of ordinary skill in the art should appreciate that various changes may be made in form and detail without departing from the spirit and scope of the disclosure as defined by the appended claims.

What is claimed is:

1. A Lactobacillus plantarum LP33 with an accession number of CCTCC NO: M2019594.

2.-4. (canceled)

5. A pharmaceutical composition for promoting lead excretion, wherein the pharmaceutical composition comprises a pharmaceutically effective dose of Lactobacillus plantarum LP33 with an accession number of CCTCC NO: M 2019594.

6. A food or a health product for promoting lead excretion, wherein the food or the health product comprises Lactobacillus plantarum LP33 with an accession number of CCTCC NO: M2019594.

7.-9. (canceled)


SEQUENCE LISTING (25 sequences)

SEQ ID NO: 1 - DNA, 1384 bp, Lactobacillus plantarum LP33
tgattggtgcttgcatcatgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcgg

SEQ ID NO: 2 - DNA, 22 bp, Artificial Sequence
  Primer, GAPDH-F
aagttcaacggcacagtcaagg

SEQ ID NO: 3 - DNA, 22 bp, Artificial Sequence
  Primer, GAPDH-R
acgccagtagactccacgacat

SEQ ID NO: 4 - DNA, 22 bp, Artificial Sequence
  Primer, Cyp7a1-F
gctgagggattgaagcacaaga

SEQ ID NO: 5 - DNA, 21 bp, Artificial Sequence
  Primer, Cyp7a1-R
gatgcccagagaatagcgagg

SEQ ID NO: 6 - DNA, 23 bp, Artificial Sequence
  Primer, Cyp8b1-F
cagatttgacctacttttcccca

SEQ ID NO: 7 - DNA, 22 bp, Artificial Sequence
  Primer, Cyp8b1-R
cccaaccagttacttatgccgt

SEQ ID NO: 8 - DNA, 19 bp, Artificial Sequence
  Primer, Shp-F
tcttcctgcttgggttggc

SEQ ID NO: 9 - DNA, 20 bp, Artificial Sequence
  Primer, Shp-R
gagggttgtggtgggtctgg

SEQ ID NO: 10 - DNA, 20 bp, Artificial Sequence
  Primer, Bsep-F
ccctgtgaaggcatggtgac

SEQ ID NO: 11 - DNA, 23 bp, Artificial Sequence
  Primer, Bsep-R
ggatgttttctgcgatagtggtg

SEQ ID NO: 12 - DNA, 22 bp, Artificial Sequence
  Primer, Ntcp-F
ttacggctacctcctccctgat

SEQ ID NO: 13 - DNA, 20 bp, Artificial Sequence
  Primer, Ntcp-R
tccatgctgatggtgcgtct

SEQ ID NO: 14 - DNA, 21 bp, Artificial Sequence
  Primer, Mrp2-F
cagatgaggaggtttggaggg

SEQ ID NO: 15 - DNA, 23 bp, Artificial Sequence
  Primer, Mrp2-R
caggaccaggattttggattttc

SEQ ID NO: 16 - DNA, 23 bp, Artificial Sequence
  Primer, Abst-F
aacttcaatgccattctcagcac

SEQ ID NO: 17 - DNA, 20 bp, Artificial Sequence
  Primer, Abst-R
tgacagaggaagcccacgaa

SEQ ID NO: 18 - DNA, 21 bp, Artificial Sequence
  Primer, OstA-F
cagccctccattttctccatc

SEQ ID NO: 19 - DNA, 24 bp, Artificial Sequence
  Primer, OstA-R
ccaaccttgttatctttctttcga

SEQ ID NO: 20 - DNA, 22 bp, Artificial Sequence
  Primer, Fxr-F
atttacaagccacggacgagtt

SEQ ID NO: 21 - DNA, 24 bp, Artificial Sequence
  Primer, Fxr-R
gttggaataataggacgaggagga

SEQ ID NO: 22 - DNA, 21 bp, Artificial Sequence
  Primer, Fgf15-F
ccaactgcttcctgcgtatcc

SEQ ID NO: 23 - DNA, 21 bp, Artificial Sequence
  Primer, Fgf15-R
cgagtagcgaatcagcccgta

SEQ ID NO: 24 - DNA, 19 bp, Artificial Sequence
  Primer, Fgfr4-F
aacgaggaccccaagaccc

SEQ ID NO: 25 - DNA, 21 bp, Artificial Sequence
  Primer, Fgfr4-R
tggacagcggaatttgacagt